(19) United States
(12) Patent Application Publication
(10) Pub. No.: US 2012/0186509 A1
(43) Pub. Date: Jul. 26, 2012

(54) ADMINISTRATION SET FLOW CONDITION MONITOR

(76) Inventors: Zoran Milijasevic, Bayview (AU); Richard J. Parkinson, Darlinghurst (AU)

(21) Appl. No.: 13/386,749

(22) PCT Filed: Jul. 27, 2010
(86) PCT No.: PCT/AU2010/000940

§ 371 Date: Apr. 3, 2012

(30) Foreign Application Priority Data
Jul. 30, 2009 (AU) 2009903559

(51) Int. Cl. G01F 1/34; G01F 1/00

(52) U.S. Cl. 116/266; 116/264

(57) ABSTRACT

An administration set flow condition monitor (30) includes a body member (32) configured to communicate with a fluid conduit (12) of the administration set (10). A flow monitoring mechanism (40) is carried by the body member (32) for indicating flow in the fluid conduit (12) of the administration set (10). An enunciator (56, 58) is associated with the flow monitoring mechanism (40) to indicate flow condition in the fluid conduit (12) of the administration set (10).

### CROSS-REFERENCE TO RELATED APPLICATIONS

The present application claims priority from Australian Provisional Patent Application No. 2009903559 dated 30 Jul. 2009, the contents of which are incorporated herein by reference in their entirety.

### FIELD

This disclosure relates, generally, to an administration set and, more particularly, to an administration set flow condition monitor and to an administration set including a flow condition monitor.

### BACKGROUND

In gravity-type administration sets, a drip chamber is provided immediately below the fluid reservoir to allow a nurse quickly and visually to check that the intravenous fluid is indeed flowing. It also enables the nurse visually to set the flow rate by adjusting a flow controller such as a roller clamp. The nurse sets the flow rate by counting the number of drops (which generally have a known volume) over a set period of time. Further, the drip chamber traps air and inhibits entry of air into the venous system.

In sets where pressure is imparted to the fluid, such as in an ambulatory system. there is usually no need for a drip chamber. Air is expelled through an air externalisation filter. The absence of a drip chamber means that there is no way of instantly determining that there is fluid flow.

### SUMMARY

In a first aspect there is provided an administration set flow condition monitor which includes

a body member configured to communicate with a fluid conduit of the administration set;

a flow monitoring mechanism carried by the body member for indicating flow in the fluid conduit of the administration set; and

an enunciator associated with the flow monitoring mechanism to indicate flow condition in the fluid conduit of the administration set.

The body member may include an attachment formation for attachment to the fluid conduit of the administration set. In an embodiment, the body member may be mountable about the conduit. In another embodiment, the body member may be mountable in line in the fluid conduit. In still another embodiment, the body member may be formed integrally with the fluid conduit as a one piece unit.

The body member may include a flow control device arranged within the body member, the flow monitoring mechanism being responsive to flow through the flow control device for indicating flow in the fluid conduit of the set.

The flow monitoring mechanism may be selected from the group consisting of mechanical devices, thermal devices, electronic devices, aural devices (including ultrasonic devices), and combinations of the foregoing. Further, the mechanical devices may include pressure measuring devices and visual, movement indicating devices.

The flow monitoring mechanism may be configured to indicate flow rate through the conduit of the administration set. The flow rate may be a current, or instantaneous flow rate, it may be an average flow rate or it may indicate that flow is within a range such as, for example, 0-5 ml/hr, 5-20 ml/hr, or the like.

The enunciator is at least one of a visual unit, an audible unit and a combination of the foregoing. Further, the enunciator may include a discernible unit which is discernible remotely from the location of the administration set. At least the discernible unit may emit both an audible and a visual alarm signal. The signal may be transmitted via wire, radio frequency such as Bluetooth, infrared, or other means.

In a second aspect there is provided an administration set which includes a flow condition monitor, as described above.

Preferably the administration set has no drip chamber.

### BRIEF DESCRIPTION OF DRAWINGS

Embodiments are now described by way of example with reference to the accompanying drawings in which:—

FIG. 1 shows a front view of an embodiment of an administration set including an embodiment of a flow condition monitor;

FIG. 2 shows a schematic, sectional side view of the embodiment of the flow condition monitor used in the administration set of FIG. 1;

FIG. 3 shows a side view of the flow condition monitor of FIG. 2;

FIG. 4 shows a schematic, sectional side view of another embodiment of a flow condition monitor;

FIG. 5 shows a schematic, sectional side view of a further embodiment of a flow condition monitor; and

FIG. 6 shows a schematic, sectional side view of still another embodiment of a flow condition monitor.

### DETAILED DESCRIPTION OF EXEMPLARY EMBODIMENTS

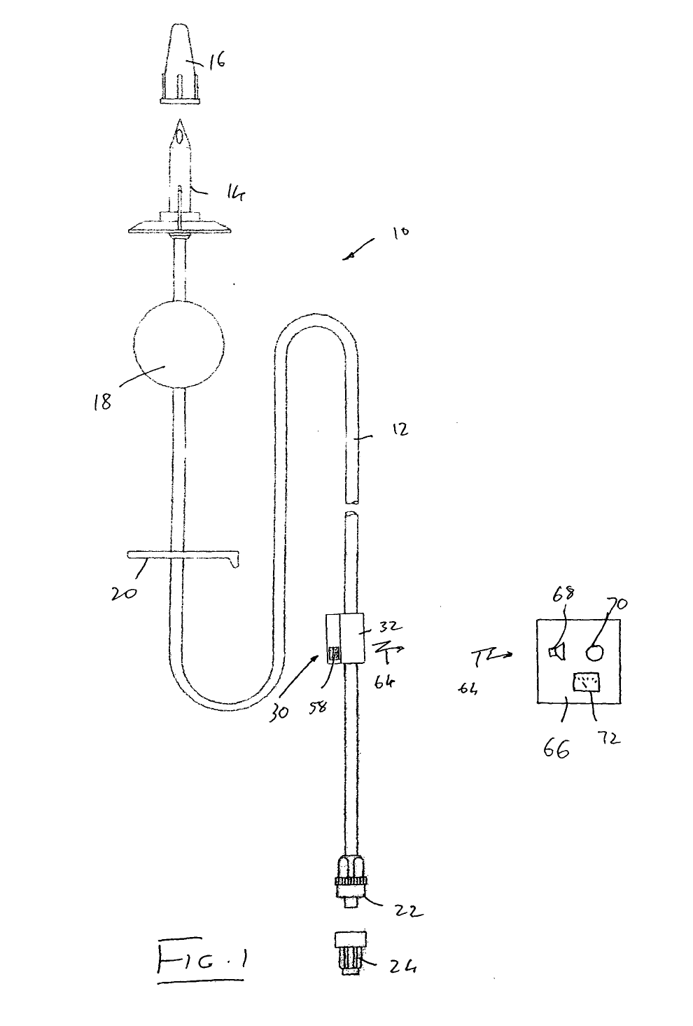

Referring initially to FIG. 1 of the drawings, reference numeral 10 generally designates an embodiment of an administration set. The administration set 10 includes tubing 12 depending from a spike 14. The spike 14 is, in use, received in a reservoir of fluid (not shown), such as a flexible bag, to be dispensed intravenously. The spike 14 is closed off by an end cap 16. An air externalisation filter 18 is arranged in the tubing 12, downstream of the spike 14. An optional slide clamp 20 is provided on the tubing 12 to block flow of fluid in the tubing 12.

An end of the tubing 12, opposite the end having the spike 14, includes a locking member in the form of a Luer lock 22 with a cap 24 being provided to close off the lock 22.

An embodiment of a flow condition monitor, generally by the reference numeral 30, is mounted in line in the tubing 12 and communicates with an interior of the tubing 12 to monitor flow of fluid in the tubing 12. In other embodiments, the flow condition monitor could be mounted about the tubing 12 or could be formed integrally with the tubing 12.

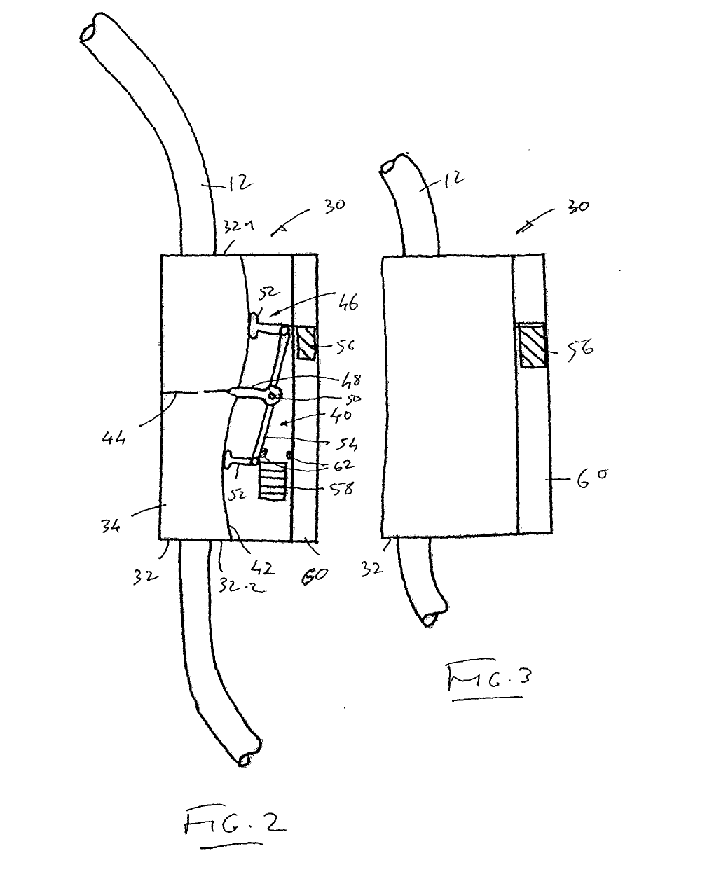

An embodiment of the flow condition monitor 30 is illustrated in greater detail in FIGS. 2 and 3 of the drawings. The flow condition monitor 30 includes a body member, or housing, 32 attachable to the tubing so that an interior 34 of the housing 32 communicates with the interior of the tubing 12 of the administration set 10.

The flow condition monitor 30 includes a flow monitoring mechanism 40 arranged within the interior 34 of the housing 32. In this embodiment, the flow monitoring mechanism 42 includes a resiliently flexible membrane 42 arranged within the interior 34 of the housing 32 to extend from a first, upstream end 32.1 of the housing 32 to a second, downstream end 32.2 of the housing 32. The membrane 42 is of a resiliently flexible material such as an elastomeric material. The membrane 42 is arranged on opposed sides of a flow control device in the form of an orifice plate 44 arranged substantially centrally within the interior 34 of the housing 32.

The membrane 42 cooperates with a sensing arrangement 46 arranged within the interior 34 of the housing on opposite sides of the orifice plate 44. In this embodiment, the sensing arrangement 46 comprises an anchor 48 defining a pivot point 50. A pair of spaced feet 52 bear against the membrane 42, one foot 52 being arranged on each side of the anchor 48. The feet 52 are arranged at opposed ends of a beam 54 pivotally mounted to the anchor 48 via the pivot point 50.

The flow condition monitor 30 comprises a local enunciator in the form of a pair of flags 56 and 58. The flag 56 is associated with the upstream foot 52 and the flag 58 is associated with the downstream foot 52. The flags 56, 58 are mounted to the feet at the hinged connection of each foot 52 to its associated end of the beam 54. Further, the flags 56 and 58 are differently coloured so that a user can readily discern the condition of flow in the tubing 12. For this purpose, the housing 32 includes a window 60 through which at least one of the flags 56 or 58 is visible as shown in FIG. 3 of the drawings.

In use, when fluid is flowing through the tubing 12 and, as a result, through the interior 34 of the housing 32, a higher pressure is generated at an upstream end of the orifice plate 44 then at a downstream end. As a result, the upstream end of the membrane 42 bulges outwardly urging the flag 56 into the window 60 of the housing 32, as shown in FIG. 3 of the drawings while the downstream end of the membrane 42 is substantially unstressed. Should a blockage occur, a higher pressure will build up downstream of the orifice plate 44. This causes the downstream portion of the membrane 42 to bulge outwardly. The upstream end of the membrane 42 is substantially unstressed and the flag 58 is urged into the window 60.

Further, when fluid flow stops, for example, as a result of the fluid reservoir of the set 10 being empty, the pressure across the orifice plate 44 equalises so that at least a portion of each flag 56, 58 is visible through the window 60. In this way, the condition of fluid flow in the administration set 10 can be determined by a quick visual inspection.

It will be appreciated that the sensing arrangement 46 is localised so that medical staff can only determine whether or not there is fluid flow in the set 10 by being present in the vicinity of the set 10. However, it would be desirable if a user could also determine, remotely, if the administration set 10 is operating normally and that there is normal fluid flow in the set 10. For this purpose, at least one pair of normally open contacts 62 is arranged within the interior of the housing 32, the contacts being associated with the flag 58. When there is a blockage in the tubing 12 resulting in a higher pressure downstream of the orifice plate 44 and the flag 58 is displaced into the window 60, the contacts 62 close. A signal 64 (FIG. 1) is generated and is sent to a remote enunciator 66. The enunciator 66 generates a discernible alarm. As illustrated, the enunciator 66 includes a speaker 68 to output an audible alarm as well as a visual indicator such as a lamp 70 to output a visual alarm. In this way, for example at a nurses station, the medical staff can take action when a blockage occurs.

If desired, a further set of contacts (not shown) can be included in the housing 32 so that, when fluid flow ends, and the sensing arrangement 46 adopts its quiescent state where both flags 56 and 58 are at least partially visible in the window 60, a further signal is generated resulting in the generation of a further alarm at the enunciator 66, the further alarm possibly having different characteristics such as a different cadence or frequency of sound and/or a different colour of light.

The enunciator 66 communicates with the sensing arrangement 46 in any of a number of ways such as by being wired to the sensing arrangement 46, wirelessly, a short range radio technology such as Bluetooth, or the like.

While the above embodiment has been described with reference to the flow monitoring mechanism being in the form of a membrane 42 arranged within the housing 32, it will be appreciated that, instead, the flow monitoring mechanism could be a balloon-type arrangement. Instead, the flow monitoring mechanism could constitute one wall of the housing 32 with the sensing arrangement 46 being arranged externally of the housing 32.

It is also possible to use the flow condition monitor 30 to determine the flow rate of fluid in the tubing 12. This is effected by knowing the elastic properties of the membrane 42 and the characteristics of the orifice plate 44. A position sensing arrangement (not shown) which is responsive to the position of the upstream foot 52, is connected to the sensing arrangement 46 so that, as the upstream foot 52 is displaced, flow rate can be inferred. The flow rate is sent via an appropriate signal to the enunciator 66 to be displayed on a display 72 for medical staff.

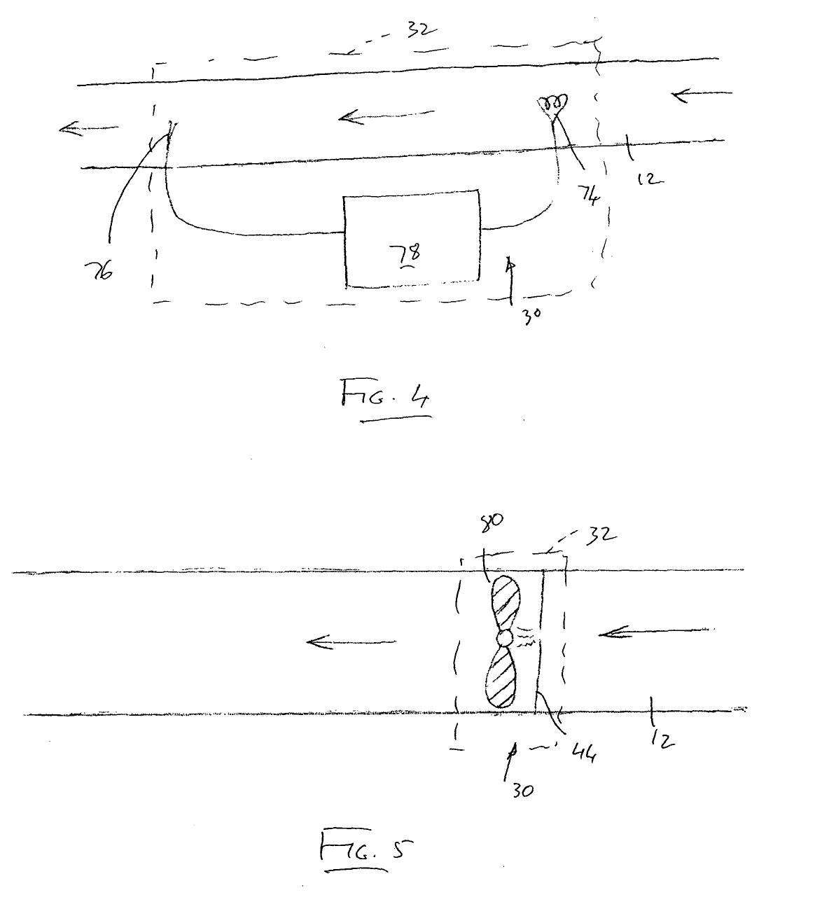

In FIG. 4 of the drawings, a further embodiment of the flow condition monitor 30 is illustrated. The monitor 30 of this embodiment is a temperature sensitive device and includes a heater 74 incorporating a temperature sensor, for heating an upstream portion of fluid flowing in the tubing 12 of the set 10. The flow condition monitor 30 further includes a temperature sensor 76 for monitoring the downstream temperature of the fluid flowing in the tubing 12. Under normal flow conditions, the downstream temperature, as measured by the sensor 76, will be lower than the upstream temperature of the fluid. The difference in temperature is an indication of the flow rate. If there is a blockage, the temperature equalises and a control unit 78 of the monitor 30 generates an appropriate alarm to enable action to be taken.

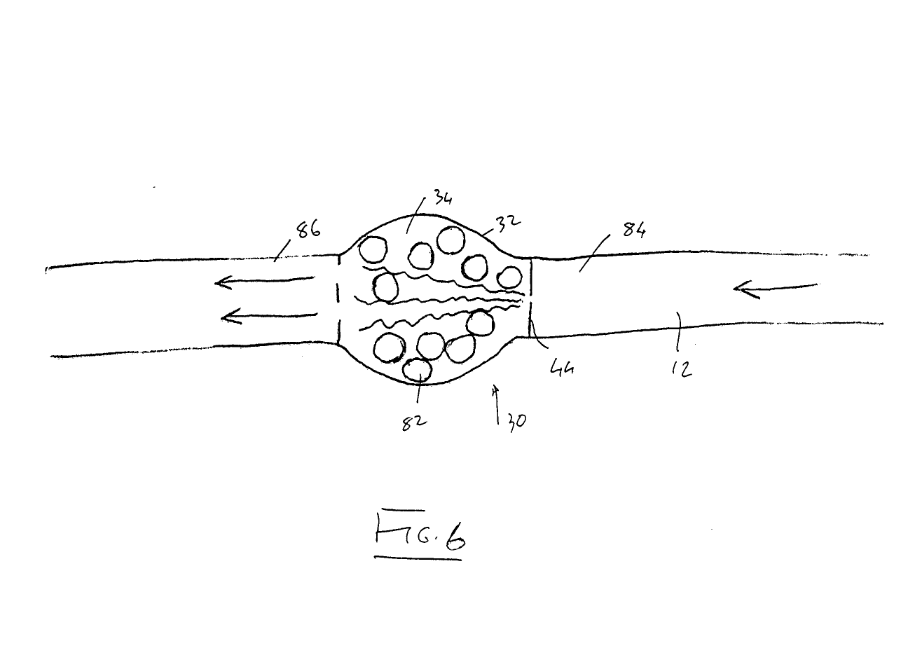

In FIG. 5 of the drawings, the fluid condition monitor 30 is a mechanical device. More particularly, the monitor 30 comprises a propeller 80 arranged downstream of the orifice plate 44 in the tubing 12. The propeller 80 provides a ready, visual indication of flow through the orifice plate 44. Preferably, the propeller 80 is a low friction, lightweight device to accommodate low flow rates in the tubing 12.

In FIG. 6 of the drawings, the flow condition monitor 30 comprises a plurality of balls 82 arranged in the housing 32, downstream of the orifice plate 44. In the administration set 10, laminar flow occurs in a part 84 of the tubing 12 upstream of the orifice plate 44. As the fluid passes through the orifice plate, the flow changes to turbulent flow in the interior 34 of the housing 32. Once the fluid passes through the housing 32 into a downstream part 86 of the tubing 12, the flow reverts to laminar flow. The presence of turbulent flow in the interior 34 of the housing 32 agitates the balls 82 contained within the interior 34 of the housing 32 providing a ready, visual indication of the flow of fluid in the tubing 12. It will be appreciated that, should a blockage occur or the fluid receptacle empty, the balls 82 will adopt a rest condition in the interior 34 of the housing 32.

It is therefore an advantage of the disclosed embodiments that a flow condition monitor 30 is provided which provides a ready indication to medical staff of flow in an administration set 10. In certain embodiments, low cost flow condition monitors 30 are provided which would find use in poorer areas or less developed areas of the world. By early alarm or warning to medical staff, remedial action can be taken when blockage occurs. It will be appreciated that this is of increased importance in the case of arterial infusion where early warning of cessation of flow is required.

It will be appreciated by persons skilled in the art that numerous variations and/or modifications may be made to the invention as shown in the specific embodiments without departing from the scope of the disclosure as broadly described. The present embodiments are, therefore, to be considered in all respects as illustrative and not restrictive.

1. An administration set flow condition monitor which includes
a body member configured to communicate with a fluid conduit of the administration set;
a flow monitoring mechanism carried by the body member for indicating flow in the fluid conduit of the administration set; and
an enunciator associated with the flow monitoring mechanism to indicate flow condition in the fluid conduit of the administration set.

2. The monitor of claim 1 in which the body member includes an attachment formation for attachment to the fluid conduit of the administration set.

3. The monitor of claim 1 in which the body member is formed integrally with the fluid conduit as a one piece unit.

4. The monitor of claim 1 in which the body member includes a flow control device arranged within the body member, the flow monitoring mechanism being responsive to flow through the flow control device for indicating flow in the fluid conduit of the set.

5. The monitor of claim 1 in which the flow monitoring mechanism is selected from the group consisting of mechanical devices, thermal devices, electronic devices, aural devices, Doppler shift devices and combinations of the foregoing.

6. The monitor of claim 5 in which the mechanical devices include pressure measuring devices and visual, movement indicating devices.

7. The monitor of claim 1 in which the flow monitoring mechanism is configured to indicate flow rate through the conduit of the administration set.

8. The monitor of claim 1 in which the enunciator is at least one of a visual unit, an audible unit and a combination of the foregoing.

9. The monitor of claim 8 in which the enunciator includes a discernible unit which is discernible remotely from the location of the administration set.

10. An administration set which includes a flow condition monitor as claimed in claim 1.

11. The administration set of claim 10 which has no drip chamber.
